(19) United States
(12) Patent Application Publication
(10) Pub. No.: US 2019/0374291 A1
(43) Pub. Date: Dec. 12, 2019

(54) SYSTEM AND METHODS FOR INTERVENTIONAL IMAGE NAVIGATION AND IMAGE REGISTRATION REFINEMENT

(71) Applicant: Clear Guide Medical, Inc., Baltimore, MD (US)

(72) Inventors: Philipp Jakob Stolka, Baltimore, MD (US); Ehsan Basafa, Cockeysville, MD (US)

(73) Assignee: Clear Guide Medical, Inc., Baltimore, MD (US)

(21) Appl. No.: 16/463,189

(22) PCT Filed: Nov. 21, 2017
(86) PCT No.: PCT/US2017/062883

§ 371 Date: May 22, 2019

Related U.S. Application Data
(60) Provisional application No. 62/426,024, filed on Nov. 23, 2016.

(51) Int. Cl. A61B 34/20; A61B 8/08; A61B 5/055; A61B 6/03; A61B 8/00; G06T 11/60; G06T 7/00

(52) U.S. Cl. CPC A61B 34/20; A61B 8/0841; A61B 5/055; A61B 6/032; A61B 8/463; A61B 2090/3762; G06T 11/60; G06T 7/0012; A61B 2034/2051; A61B 2034/2063; A61B 2034/2065; A61B 8/5261

(57) ABSTRACT

A system and a method for surgical image guidance are described herein. The system includes a first imaging device and an image processing system operatively connected to the first imaging device. The image processing system is configured to: receive real-time image data from the first imaging device; receive secondary image data from a second imaging device; produce enhanced composite image data by improving an alignment of physical structures in a real-time image. The image processing system is configured to operate in an unlocked mode in which the real-time image is free to move relative to the secondary image and a locked mode wherein the real-time image and the secondary image are locked relative to each other to prevent relative movement therebetween. The imaging device is configured to be able to provide information to the image processing system when the image processing system is operating in the unlocked mode.

### CROSS-REFERENCE TO RELATED APPLICATIONS

The present patent application claims priority benefit from U.S. Provisional Patent Application No. 62/426,024 filed on Nov. 23, 2016, the entire content of which is incorporated herein by reference.

### BACKGROUND

### 1. Technical Field

This disclosure generally relates to image systems for navigating surgical medical devices. More particularly, this disclosure relates to systems and methods for navigating interventional instrumentation, such as surgical needles, and refining registration between image modalities.

### 2. Discussion of Related Art

In the course of performing a surgical operation or intervention, a medical practitioner (e.g., a surgeon) may use various operating instruments to perform various operations such as needle biopsies, tumor ablations, catheter insertion, orthopedic interventions, etc. These instruments may use several types of image data to aid the medical practitioner or operator in inserting the instrument into a desired location within a patient's body. Typically, the medical practitioner inserts these instruments into a patient's body at very specific locations, orientations, and depths to reach predetermined target areas in order to perform an instrument-specific action or function, which may include tissue sampling, heating, cooling, liquid deposition, suction, or serving as a channel for other objects.

Medical navigation systems based on a variety of different tracking technologies (mechanical, infrared-optical, electromagnetic, etc.) have existed for a long time, and help with aligning patient, imaging data, targets, and instruments. However, the known medical navigation systems remain inaccurate, inconvenient, and ineffective in providing real-time data and guidance in inserting and moving interventional instruments.

Therefore, a need remains for improved systems and methods for interventional image navigation and image registration refinement.

### SUMMARY OF THE DISCLOSURE

An aspect of the present disclosure is to provide a system for surgical image guidance. The system includes a first imaging device, and an image processing system operatively connected to the first imaging device. The image processing system is configured to: 1) receive real-time image data of a patient from the first imaging device; 2) receive secondary image data from a second imaging device; 3) produce composite image data based on the real-time image data and the secondary image data; and 4) produce enhanced composite image data by improving an alignment of physical structures in a real-time image based on the real-time image data with corresponding physical structures in a secondary image based on the secondary image data. The image processing system is further configured to operate in an unlocked mode in which the real-time image is free to move relative to the secondary image and a locked mode wherein the real-time image and the secondary image are locked relative to each other to prevent relative movement therebetween. The imaging device is configured to be able to provide information to the image processing system to cause the image processing system to operate in the unlocked mode to enable movement of the real-time image relative to the secondary image.

Another aspect of the present disclosure is to provide a method for surgical image guidance. The method includes receiving, by an image processing system, real-time image data of a patient from the first imaging device; receiving, by the image processing system, secondary image data from a second imaging device; producing, by the image processing system, composite image data based on the real-time image data and the secondary image data; and producing, by the image processing system, enhanced composite image data by improving an alignment of physical structures in a real-time image based on the real-time image data with corresponding physical structures in a secondary image based on the secondary image data. The method further includes receiving, by the image processing system, a command to permit moving the real-time image relative to the secondary image when the image processing system is operating in an unlocked mode in which the real-time image is free to move relative to the secondary image, and receiving, by the processing system, a command to put the processing system in a locked operating mode so as to prevent relative movement between the real-time image and the secondary image.

A further aspect of the present disclosure is to provide a non-transitory processor-readable medium having instructions stored thereon, which when executed by one or more processors, cause the one or more processors to implement the above method.

### BRIEF DESCRIPTION OF THE DRAWINGS

The present disclosure, as well as the methods of operation and functions of the related elements of structure and the combination of parts and economies of manufacture, will become more apparent upon consideration of the following description and the appended claims with reference to the accompanying drawings, all of which form a part of this specification. Like reference numerals designate corresponding parts in the various figures. The drawings are for the purpose of illustration and description only and are not intended as a definition of the limits of the invention.

FIG. 1 depicts an example of an ultrasound system (Clear Guide SCENERGY system) having an ultrasound probe having mounted thereon a tracking device (e.g., one or more cameras) for tracking a position of the ultrasound probe on a patient's body;

FIG. 2 depicts the insertion of a needle into a patient's body by a health practitioner while using an ultrasound system (Clear Guide Medical “SCENERGY” system) having the one or more cameras to track the position of the needle relative to patient's body, the ultrasound system providing an image displaying information about a path of the needle insertion within the patient's body;

FIG. 3 shows a plurality of VisiMARKERs that are applied in an irregular fashion around the intervention area in a body of a patient;

FIG. 4 depicts a camera sweep wherein the camera captures a plurality of images including the VisiMARKERS;

FIG. 5 illustrates a position of the markers on the body of the patient, and shows images corresponding to one image modality (e.g., CT scan image modality) superposed thereon a track of a surgical device (e.g., a needle) being inserted into the body of the patient and another image modality (e.g., ultrasound scan image modality) superposed thereon a track of a surgical device (e.g., a needle) being inserted into the body of the patient;

FIG. 6A depicts an example of two superposed image modalities, one image modality representing a CT scan and the other image modality representing an ultrasound scan, according to an embodiment of the present disclosure; and

FIG. 6B depicts an example of the two superposed image modalities being optimally overlapped, according to an embodiment of the present disclosure.

### DETAILED DESCRIPTION

The present disclosure describes medical-device systems and methods that allow operators to perform navigation-assisted interventions using certain types of intervention instruments, such as needle-like instruments, and corresponding methods of optimizing registration between image modalities using visual or automatic termination criteria. Example applications may include needle biopsies, tumor ablations, catheter insertion, orthopedic interventions, and other instruments, all of which may use several types of image data. Typically, these instruments are inserted into a patient's body at very specific locations, orientations, and depths to reach predetermined target areas, where they perform an instrument-specific action or function, which may include tissue sampling, heating, cooling, liquid deposition, suction, or serving as a channel for other objects.

Clear Guide Medical has previously developed a novel visual tracking technology platform based on real-time camera-based computer vision, and embodied the tracking platform in products for ultrasound-based systems and computerized tomography (CT)-based systems for image/instrument guidance and multi-modality fusion. Certain aspects of this technology has already been described in U.S. patent application Ser. Nos. 13/648,245, 14/092,755, 14/092,843, 14/508,223, 14/524,468, 14/524,570, and 14/689,849, which are incorporated herein in their entireties for all purposes.

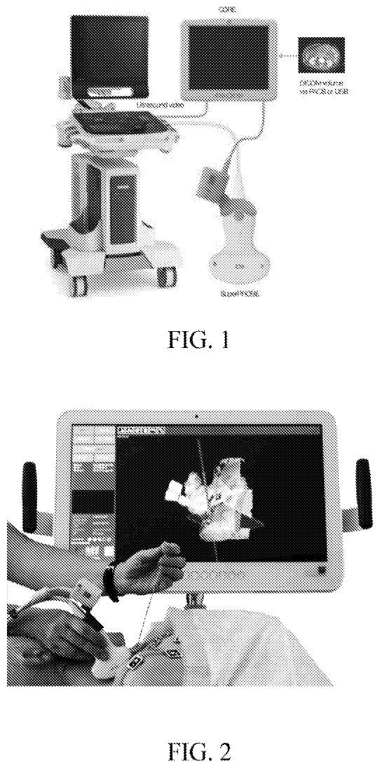

FIGS. 1 and 2 depict examples of the above-noted systems. FIG. 1. depicts an example of an ultrasound system (Clear Guide SCENERGY system) having an ultrasound probe having mounted thereon a tracking device (e.g., one or more cameras) for tracking a position of the ultrasound probe on a patient's body. One or more cameras image objects in their field of view, detect and localize reference features for registration and tracking, reconstruct their poses in camera space, and register them to corresponding objects' poses in other image data sets. As it will described further in detail in the following paragraphs, the ultrasound system can be configured in accordance with embodiments of the present disclosure for navigating interventional instrumentation, such as surgical needles, and refining registration between image modalities.

FIG. 2 depicts a health practitioner inserting a needle into a patient's body while using an ultrasound system (Clear Guide Medical “SCENERGY” system) having the one or more cameras to track the position of the needle relative to patient's body, the ultrasound system providing an image displaying information about a path of the needle insertion within the patient's body. The Clear Guide “SCENERGY” system allows fusion of registration images of ultrasound and CT devices based on skin-attached “VisiMARKER” fiducials. These multi-modality markers comprise a radiopaque layer that is automatically segmented from radiographic imaging based on its known high intensity, and a visible checkerboard-like surface pattern that allows observing cameras to localize the marker in six degrees of freedom in camera coordinates (see also Olson E., AprilTag: A robust and flexible visual fiducial system, IEEE-ICRA 2011, the entire content of which is incorporated herein by reference).

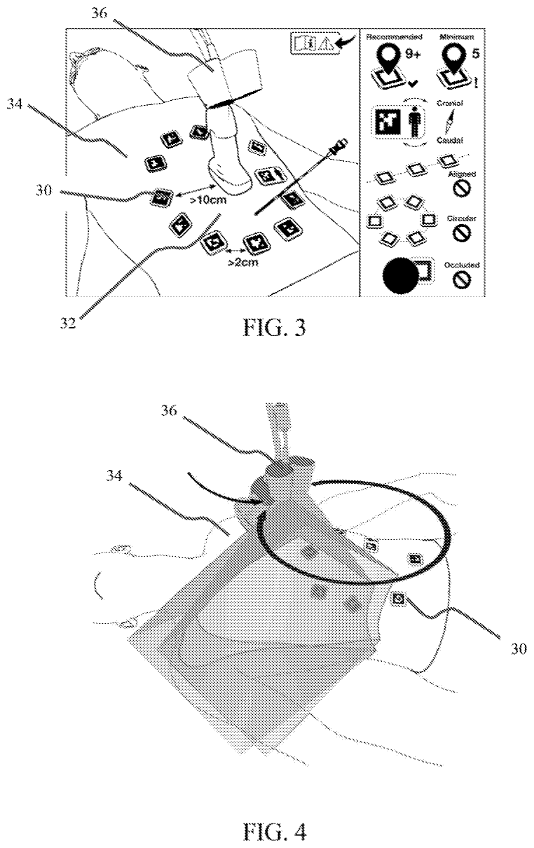

FIG. 3 shows a plurality of VisiMARKERs 30 that are applied in an irregular fashion around the intervention area 32 in a body 34 of a patient. The VisiMARKERS 30 are imaged together with the body 34 of the patient. The operator or health pratitioner moves the handheld camera head 36 in a sweeping motion above the patient surface, thus collecting marker observations.

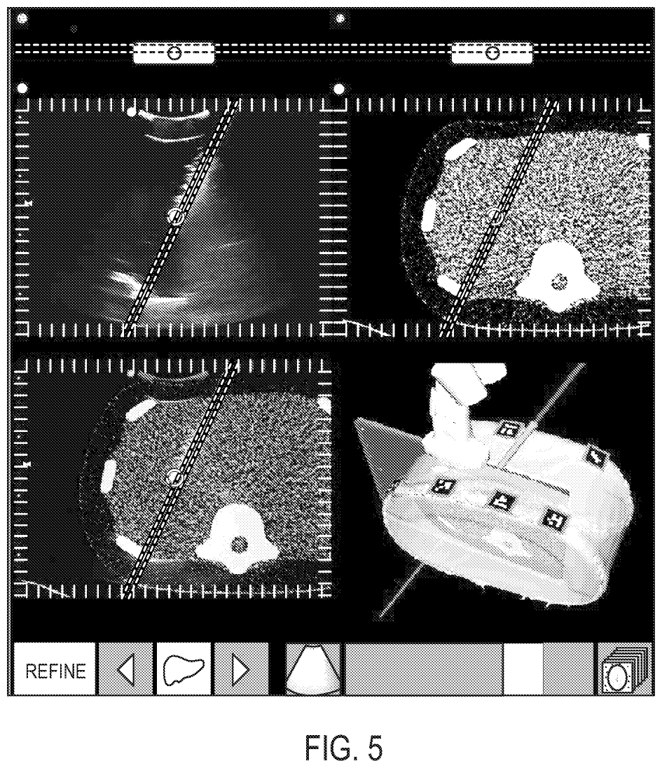

FIG. 4 depicts a camera sweep wherein the camera captures a plurality of images including the VisiMARKERS 30. Consecutive observations of marker n-tuples (for n≥2) allow the successive insertion of those markers and their relative poses into a three-dimensional (3D) mesh. This observation mesh is then automatically registered onto the marker locations auto-segmented from CT by comparing all possible alignments of both point sets and choosing the most plausible transformation M (see, U.S. patent application Ser. No. 14/689,849 to Stolka et al., the entire content of which is incorporated herein by reference). This allows the system to localize the camera 36 relative to the patient 34 (via direct observations of the markers 30), the camera 36 relative to the patient image data (via registration to the marker mesh), ultrasound relative to the camera 36 (via predetermined intrinsic device calibration), and therefore ultrasound to patient data.

The system can then continuously extract slices from the patient data that spatially correspond to the current ultrasound slice, and show both overlaid on screen in a fusion view, as shown in FIG. 5. FIG. 5, at the lower right corner, illustrates a position of the markers 30 on the body 34 of the patient. When the handheld probe with the camera 36 moves across the intervention area 32, all imaging modalities show corresponding real-time fused views. The upper right and lower left corner of FIG. 5 are shown images corresponding to one image modality (e.g., CT scan image modality) superposed thereon a track of a surgical device (e.g., a needle) being inserted into the body of the patient. In the upper left corner of FIG. 5 is shown another image modality (e.g., an ultrasound scan image modality) superposed thereon a track of a surgical device (e.g., a needle) being inserted into the body of the patient.

Manual Registration Refinement: However, the above process for performing a “Visual Sweep” registration based on the position of the markers 30 is subject to noise including patient breathing artifacts during initial imaging or the Visual Sweep, marker drift or skin shift between imaging and registration, imperfect marker observations, internal tissue shift etc. These noise sources may deteriorate the registration quality, leading to noticeable displacement between registered image data and actual relevant anatomical features as viewed by real-time imaging such as ultrasound or fluoroscopy.

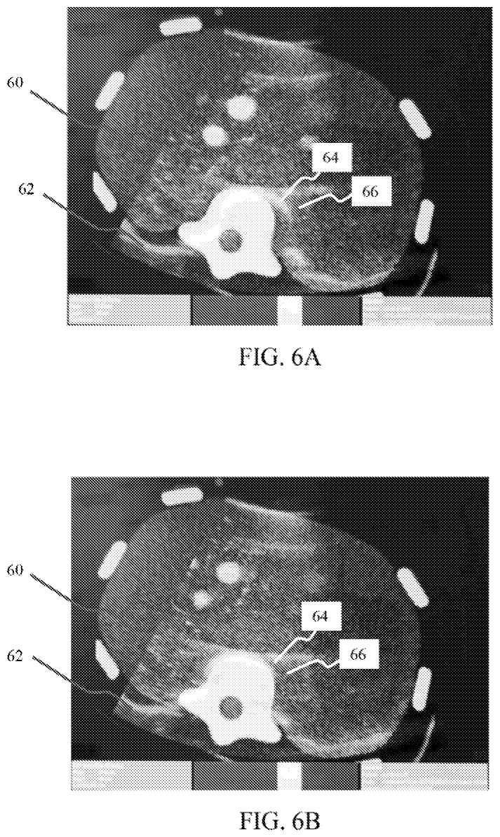

FIG. 6A depicts an example of two superposed image modalities, one image modality 60 representing a CT scan and the other image modality 62 representing an ultrasound scan, according to an embodiment of the present disclosure. As shown in FIG. 6A, the features shown in CT image 60 do not line up with corresponding features shown in ultrasound image 62. For example, a profile 64 of a backbone (simulating the backbone of a patient) shown in CT image 60 does not coincide with a profile 66 of the same backbone shown in ultrasound image 62. The two profiles 64 and 66 appear to be shifted relative to each other.

The term “real-time” imaging is used herein to mean (a) that the images are captured while the patient is imaged with the imaging system(s), (b) the time the patient is willing to wait on the imaging table, (c) the images are viewed within approximately one minute (e.g., less than 30 seconds) from capturing the image by a probe (e.g. ultrasound probe), or (d) the images are viewed within less than one second from capturing the images (i.e., relatively instantaneously).

Therefore, it may be desirable to adjust the initial, automatically determined registration matrix M to achieve better feature alignment. In an embodiment, one possible procedure to correct for misalignment is to allow an operator (e.g., medical practitioner) to perform a “manual registration refinement”, as follows:
1. The operator observes relevant feature misalignment between two fused or superposed image modalities. For example, the operator may observe misalignment of profile 64 and profile 66 of the backbone, as shown in FIG. 6A.
2. The operator “unlocks” the real-time image modality while maintaining the static image modality “frozen” in a same position. For example, the operator may unlock the ultrasound image modality 62 while the CT image modality 60 remains static in position. For example, this can be performed by activating a button, switch or other feature on the ultrasound scanner system to allow the captured ultrasound image 62 to refresh or update in real-time and move relative to the captured CT scan image modality 60 which remains fixed in position.
3. The operator systematically moves the real-time image (e.g., by moving the ultrasound probe) such that relevant features optimally overlap features in the frozen image modality. For example, the operator may move the ultrasound probe until the profile 66 shown in the captured ultrasound image modality 62 coincides or overlaps with the profile 64 shown in the captured CT scan image modality 60. FIG. 6B depicts an example of the two superposed image modalities 60 and 62 being optimally overlapped, according to an embodiment of the present disclosure. The term “optimally overlapped” is used herein to mean that the image modalities 60 and 62 appear to coincide to the best visual assessment ability of the operator.
4. The operator then “re-locks” the real-time modality (e.g., the ultrasound image modality 62) to the static image modality (e.g., the CT scan image modality 60). For example, in an embodiment, the image modalities 60 and 62 can be both allowed to resume moving together in unison while remaining locked in position relative to each other.

By using the above procedure, the operator can guide image registration optimization both by manually searching for the optimal alignment pose, and by visually assessing alignment quality.

Automatic Termination: In another embodiment, the above-described manual refinement procedure may suffer from subjectivity as it depends on a visual alignment assessment of the operator. Furthermore, the above-described manual alignment may also have usability shortcomings in that multiple interaction types during dexterous high-accuracy actions may be needed. As a result, in order to remedy the above deficiencies and further improve upon the above described manual/visual refinement procedure, a further alignment procedure is employed herein that removes the subjective visual assessment of the operator. In an embodiment, the alignment procedure includes:
1. The operator observes relevant feature misalignment between two fused or superposed image modalities.
2. The operator “unlocks” the real-time image modality (e.g., unlocks the ultrasound image modality 62) while the static image modality (e.g., the CT scan image modality 60) remains “frozen” in place.
3. The operator systematically moves the real-time image (e.g., moves the ultrasound image modality 62 by moving the ultrasound probe) such that relevant features optimally overlap corresponding ones in the frozen modality (e.g., the CT scan image modality 60).
4. The system automatically computes an image registration quality (IRQ) metric in real-time and displays the computed IRQ metric value to the operator as feedback. The operator may use the IRQ metric as guidance in the search for an optimum alignment. In an embodiment, the greater the value of IRQ achieved, the better is the alignment achieved between the two images modalities (e.g., CT image modality 60 and ultrasound image modality 62). The system continuously stores the maximum-to-date achieved IRQ value, and the corresponding alignment pose that allowed for this maximum IRQ value.
5. The system compares a first IRQ value obtained in a first attempted alignment and a second IRQ value obtained with a second subsequently attempted alignment and selects an alignment among the first attempted alignment and the second attempted alignment having the greater IRQ value among the first and second IRQ values as the best alignment to date.
6. The system repeats this procedure and select the best alignment that achieves a greater IRQ value.
7. If the system determines that, after a certain number of iterations, no improvement is possible any more, or no improvement expected any more based on the IRQ metric's trend (e.g., the IRQ value plateaued or constantly falling in subsequent attempts for alignment), or after the IRQ value has reached a certain threshold value (e.g., selected by the user), alignment refinement is automatically terminated. A previously stored pose having the maximum-to-date achieved IRQ value (or the initial registration if no improvement was found) is selected as being the optimum alignment.
8. The system automatically “re-locks” the real-time modality (e.g., the ultrasound image modality 62) to the static image modality (e.g., the CT scan image modality 60). For example, in an embodiment, the image modalities 60 and 62 can be both allowed to resume moving together in unison while remaining locked in position relative to each other. The term “automatically” is used herein to indicate that the system performs a certain function without manual interference from the user or operator.
9. Alternatively, the system provides an audio or visual feedback to the operator to inform the operator that the optimum alignment is reached. The operator then can manually “re-lock” the real-time modality (e.g., the ultrasound image modality 62) to the static image modality (e.g., the CT scan image modality 60). For example, in an embodiment, the image modalities 60 and 62 can be both allowed to resume moving together in unison while remaining locked in position relative to each other.

In the above paragraphs, it is described that the maximum value of the IRQ determines the best or optimum alignment between the images modalities. However, as it can be appreciated, in another embodiment, another type of IRQ metric can also be selected or generated such that the smaller the value of IRQ achieved, the better is the alignment achieved between the two images modalities (e.g., CT image modality 60 and ultrasound image modality 62). In this case, instead of tracking or following the maximum value of the IRQ metric, the minimum value of the IRQ metric provides the indication when the optimum alignment between the image modalities is achieved.

This approach has the benefit of relieving the operator of the alignment assessment burden. The system computes the image registration quality (IRQ) metric by correlating the respective current modality images (real-time and “frozen” static slices). Depending on combination of image modalities, the IRQ function may be straightforward image correlation for identical modalities, but may require pre-processing in other cases. For example, ultrasound is a boundary-based modality, whereas CT is a volume-based modality. Initially, the CT scanner's vertical gradient image may be computed (to approximate the ultrasound's top-down insonification and the resultant highlighting of horizontal tissue interfaces with large acoustic impedance jumps). Then, a correlation or better mutual-information-based (see, also Wells et al., “Multi-Modal Volume Registration by Maximization of Mutual Information,” Medical Image Analysis, 1996, the entire content of which is incorporated herein by reference) may be used to estimate alignment quality.

Since an initial visual sweep registration is almost guaranteed to result in a registration very close to the global optimum of the IRQ function, one may assume that the manual sampling of the pose space by the operator consistently results in higher values closer to the global optimum, and in lower values further away. This safely allows the system to automatically terminate the optimization and return the optimum-to-date pose as the result.

This approach may be also applied in cases where the real-time imaging modality is substituted or combined by one or more other image modalities or data sets (such as other CT, CBCT, MRI, etc. volumes). The manual refinement may be performed with real-time imaging against any other operator or automatically selected static volumes (e.g., by automatically choosing the lowest-quality matched data set for unlocking), as well as with static volumes against other static volumes, using similar interaction and computation approaches as outlined above.

As it can be appreciated from the above paragraphs, in an embodiment of the present disclosure, there is provided a system for surgical image guidance. The system includes a first imaging device and an image processing system operatively connected to the first imaging device. The image processing system is configured to: 1) receive real-time image data of a patient from the first imaging device; 2) receive secondary image data from a second imaging device; 3) produce composite image data based on the real-time image data and the secondary image data; and 4) produce enhanced composite image data by improving an alignment of physical structures in a real-time image based on the real-time image data with corresponding physical structures in a secondary image based on the secondary image data. The image processing system is further configured to operate in an unlocked mode in which the real-time image is free to move relative to the secondary image and a locked mode wherein the real-time image and the secondary image are locked relative to each other to prevent relative movement therebetween. The imaging device is configured to be able to provide information to the image processing system to cause the image processing system to operate in the unlocked mode to enable movement of the real-time image relative to the secondary image.

In an embodiment, the system may further include a display device configured to receive and display the composite image data. In an embodiment, the first imaging device can be an ultrasound device and the second imaging device can be a CT scan device or an Mill device or a three-dimensional medical imaging device.

In an embodiment, the image processing system can be configured to allow correcting of a misalignment between the real-time image data relative to the secondary image data until an operator determines that the real-time image data coincides with the secondary image data.

In an embodiment, the image processing system may include an input device configured to receive an input from an operator to put the image processing system in the unlocked mode to allow the real-time image to update and move in position relative to the secondary image. In an embodiment, the input device can be further configured to receive an input from the operator to put the image processing system in the locked mode to prevent relative movement between the real-time image and the secondary image.

In an embodiment, the image processing system can be configured to compute an image registration quality (IRQ) metric continuously. The IRQ metric quantifies a degree of the alignment of the physical structures in the real-time image with the corresponding physical structures in the secondary image. In an embodiment, the image processing system can be configured to determine whether the IRQ metric meets a predetermined threshold value or a dynamically determined threshold value, and provide, based on the determination, a feedback signal to an operator or to automatically put the image processing system in the locked mode to prevent relative movement between the real-time image and the secondary image. In an embodiment, the image processing system can be configured to display the IRQ metric as feedback. In an embodiment, the dynamically determined threshold value is a maximum or a minimum IRQ value. In an embodiment, the image processing system can be configured to store the IRQ metric and a corresponding alignment pose that achieved the IRQ metric. In an embodiment, the image processing system can be configured to compare a first IRQ metric obtained in a first attempted alignment pose with a second IRQ metric obtained with a second attempted alignment pose, and to store as an alignment pose the first attempted alignment pose or the second attempted alignment pose that achieved a best IRQ value among the first IRQ metric and the second IRQ metric. For example, the best IRQ metric corresponds to a higher IRQ metric in the first and second IRQ metrics, a lower IRQ metric in the first and second IRQ metrics, or an IRQ metric among the first and second IRQ metrics that is closest to the predetermined threshold value.

In an embodiment, the image processing system can be configured to automatically lock an alignment between the physical structures in the real-time image with the corresponding physical structures in the secondary image, if, after a certain number of comparison iterations, no improvement in alignment is possible any more, or no improvement in alignment is expected any more based on a trend of the IRQ metric, or after the IRQ metric has reached the predetermined threshold value, and select a previously stored alignment pose having a best achieved IRQ metric as an optimum alignment pose. For example, the IRQ metric can be determined by performing a two-dimensional correlation between the real-time image data and the corresponding secondary image data. The two-dimensional cross-correlation between the real-time image data and the corresponding secondary image data can be performed according to the following equation:

$${r}_{ij} = \frac{{\sum }_{m} {\sum }_{n} [ f ( m + i , n + j ) - \overline{f} ] [ g ( m , n ) - \overline{g} ]}{\sqrt{{\sum }_{m} {\sum }_{n} {[ f ( m , n ) - \overline{f} ]}^{2} {\sum }_{m} {\sum }_{n} {[ g ( m , n ) - \overline{g} ]}^{2}}}$$

where f(m,n) and g(m,n) are respective image pixel values in the real-time image data and corresponding secondary image data at locations m,n, with i−j−0 for determining said correlation at the current alignment pose.

As it can be appreciated from the above paragraphs, in an embodiment of the present disclosure, there is also provided a method for surgical image guidance. The method includes 1) receiving, by an image processing system, real-time image data of a patient from the first imaging device; 2) receiving, by the image processing system, secondary image data from a second imaging device; 3) producing, by the image processing system, composite image data based on the real-time image data and the secondary image data; and 4) producing, by the image processing system, enhanced composite image data by improving an alignment of physical structures in a real-time image based on the real-time image data with corresponding physical structures in a secondary image based on the secondary image data. The method also includes receiving, by the image processing system, a command permit moving the real-time image relative to the secondary image when the image processing system is operating in an unlocked mode in which the real-time image is free to move relative to the secondary image. The method further includes receiving, by the processing system, a command to put the processing system in a locked operating mode so as to prevent relative movement between the real-time image and the secondary image.

In an embodiment, the method further includes allowing for correction, by the processing system, for a misalignment between the real-time image data relative to the secondary image data until an operator determines that the real-time image data coincides with the secondary image data. In an embodiment, the method includes computing, by the processing system, an image registration quality (IRQ) metric continously, the IRQ metric quantifying a degree of the alignment of the physical structures in the real-time image with the corresponding physical structures in the secondary image. In an embodiment, the method further includes determining whether the IRQ metric meets a predetermined threshold value or a dynamically determined threshold value; and providing, based on the determination, a feedback signal to an operator or to automatically put the image processing system in the locked mode to prevent relative movement between the real-time image and the secondary image. The method may also include providing the IRQ metric as feedback to the operator. In an embodiment, the dynamically determined threshold value can be a maximum or a minimum IRQ value. The method also includes storing the IRQ metric and a corresponding alignment pose that achieved the IRQ metric.

In an embodiment, the method may also include comparing a first IRQ metric obtained in a first attempted alignment pose with a second IRQ metric obtained in a second attempted alignment pose, and storing as an alignment pose the first attempted alignment pose or the second attempted alignment pose that achieved a best IRQ value among the first IRQ metric and the second IRQ metric. For example, best IRQ metric can correspond to a higher IRQ metric in the first and second IRQ metrics, a lower IRQ metric in the first and second IRQ metrics, or an IRQ metric among the first and second IRQ metrics that is closest to the predetermined threshold value.

In an embodiment, the method may also include automatically locking an alignment between the physical structures in the real-time image with the corresponding physical structures in the secondary image, if, after a certain number of comparison iterations, no improvement in alignment is possible any more, or no improvement in alignment is expected any more based on a trend of the IRQ metric, or after the IRQ metric has reached the predetermined threshold value, and selecting a previously stored alignment pose having a best achieved IRQ metric as an optimum alignment pose.

According to an embodiment of the present disclosure, there is also provided a non-transitory processor-readable medium having instructions stored thereon, which when executed by one or more processors, cause the one or more processors to implement the above method.

The foregoing detailed description of embodiments includes references to the drawings or figures, which show illustrations in accordance with example embodiments. The embodiments described herein can be combined, other embodiments can be utilized, or structural, logical and operational changes can be made without departing from the scope of what is claimed. The foregoing detailed description is, therefore, not to be taken in a limiting sense, and the scope is defined by the appended claims and their equivalents. It should be evident that various modifications and changes can be made to these embodiments without departing from the broader spirit and scope of the present disclosure. Accordingly, the specification and drawings are to be regarded in an illustrative rather than a restrictive sense.

Present teachings may be implemented using a variety of technologies. For example, certain aspects of this disclosure may be implemented using electronic hardware, computer software, or any combination thereof. Whether such elements are implemented as hardware or software depends upon the particular application and design constraints imposed on the overall system. By way of example, the electronic hardware, or any portion of electronic hardware may be implemented with a processing system that includes one or more processors. Examples of processors include microprocessors, microcontrollers, Central Processing Units (CPUs), digital signal processors (DSPs), field programmable gate arrays (FPGAs), programmable logic devices (PLDs), state machines, gated logic, discrete hardware circuits, and other suitable hardware configured to perform various functions described throughout this disclosure. One or more processors in the processing system may execute software, firmware, or middleware (collectively referred to as “software”). The term “software” shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise. In certain embodiments, the electronic hardware can also include designed application-specific integrated circuits (ASICs), programmable logic devices, or various combinations thereof. The processing system can refer to a computer (e.g., a desktop computer, tablet computer, laptop computer), cellular phone, smart phone, and so forth. The processing system can also include one or more input devices, one or more output devices (e.g., a display), memory, network interface, and so forth.

If certain functions described herein are implemented in software, the functions may be stored on or encoded as one or more instructions or code on a non-transitory computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), compact disk ROM (CD-ROM) or other optical disk storage, magnetic disk storage, solid state memory, or any other data storage devices, combinations of the aforementioned types of computer-readable media, or any other medium that can be used to store computer executable code in the form of instructions or data structures that can be accessed by a computer.

For purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

The embodiments illustrated and discussed in this specification are intended only to teach those skilled in the art how to make and use the invention. In describing embodiments of the disclosure, specific terminology is employed for the sake of clarity. However, the disclosure is not intended to be limited to the specific terminology so selected. The above-described embodiments of the disclosure may be modified or varied, without departing from the invention, as appreciated by those skilled in the art in light of the above teachings. It is therefore to be understood that, within the scope of the claims and their equivalents, the invention may be practiced otherwise than as specifically described. For example, it is to be understood that the present disclosure contemplates that, to the extent possible, one or more features of any embodiment can be combined with one or more features of any other embodiment.

We claim:

1. A system for surgical image guidance, comprising:
a first imaging device;
an image processing system operatively connected to the first imaging device, the image processing system being configured to:
receive real-time image data of a patient from the first imaging device;
receive secondary image data from a second imaging device;
produce composite image data based on the real-time image data and the secondary image data; and
produce enhanced composite image data by improving an alignment of physical structures in a real-time image based on said real-time image data with corresponding physical structures in a secondary image based on said secondary image data,
wherein the image processing system is further configured to operate in an unlocked mode in which the real-time image is free to move relative to the secondary image and a locked mode wherein the real-time image and the secondary image are locked relative to each other to prevent relative movement therebetween, and
wherein said imaging device is configured to be able to provide information to said image processing system to cause said image processing system to operate in said unlocked mode to enable movement of said real-time image relative to said secondary image.

2. The system according to claim 1, wherein the first imaging device is an ultrasound device and the second imaging device is a CT scan device, an MRI device, or a three-dimensional medical imaging device.

3. The system according to claim 1, further comprising a display device configured to receive and display the composite image data.

4. The system according to claim 1, wherein said image processing system is configured to allow correcting of a misalignment between the real-time image data relative to the secondary image data until an operator determines that the real-time image data coincides with the secondary image data.

5. The system according to claim 1, wherein the image processing system comprises an input device configured to receive an input from an operator to put the image processing system in the unlocked mode to allow the real-time image to update and move in position relative to the secondary image.

6. The system according to claim 5, wherein the input device is further configured to receive an input from the operator to put the image processing system in the locked mode to prevent relative movement between the real-time image and the secondary image.

7. The system according to claim 1, wherein the image processing system is configured to compute an image registration quality (IRQ) metric continuously, the IRQ metric quantifying a degree of the alignment of the physical structures in the real-time image with the corresponding physical structures in the secondary image.

8. The system according to claim 7, wherein the image processing system is configured to:
determine whether the IRQ metric meets a predetermined threshold value or a dynamically determined threshold value; and
provide, based on the determination, a feedback signal to an operator or automatically put the image processing system in the locked mode to prevent relative movement between the real-time image and the secondary image.

9. The system according to claim 8, wherein the image processing system is configured to provide the IRQ metric as feedback to the operator.

10. The system according to claim 8, wherein the dynamically determined threshold value is a maximum or a minimum IRQ value.

11. The system according to claim 8, wherein the image processing system is configured to store the IRQ metric and a corresponding alignment pose that achieved the IRQ metric.

12. The system according to claim 8, wherein the image processing system is configured to compare a first IRQ metric obtained in a first attempted alignment pose with a second IRQ metric obtained in a second attempted alignment pose and to store as an alignment pose the first alignment pose or the second attempted alignment pose that achieved a best IRQ value among the first IRQ metric and the second IRQ metric.

13. The system according to claim 12, wherein the best IRQ metric corresponds to a higher IRQ metric in the first and second IRQ metrics, a lower IRQ metric in the first and second IRQ metrics, or an IRQ metric among the first and second IRQ metrics that is closest to the predetermined threshold value.

14. The system according to claim 13, wherein the image processing system is configured to:
automatically lock an alignment between the physical structures in the real-time image with the corresponding physical structures in the secondary image, if, after a certain number of comparison iterations, no improvement in alignment is possible any more, or no improvement in alignment is expected any more based on a trend of the IRQ metric, or after the IRQ metric has reached the predetermined threshold value, and
select a previously stored alignment pose having a best achieved IRQ metric as an optimum alignment pose.

15. The system according to claim 7, wherein the IRQ metric is determined by performing a two-dimensional cross-correlation between the real-time image data and the corresponding secondary image data.

16. The system according to claim 15, wherein the two-dimensional cross-correlation between the real-time image data and the corresponding secondary image data is performed according to the following equation:
$${r}_{ij} = \frac{{\sum }_{m} {\sum }_{n} [ f ( m + i , n + j ) - \overline{f} ] [ g ( m , n ) - \overline{g} ]}{\sqrt{{\sum }_{m} {\sum }_{n} {[ f ( m , n ) - \overline{f} ]}^{2} {\sum }_{m} {\sum }_{n} {[ g ( m , n ) - \overline{g} ]}^{2}}}$$
where f(m,n) and g(m,n) are respective image pixel values in the real-time image data and corresponding secondary image data at locations m,n, with i=j=0 for determining said correlation at the current alignment pose.

17. A method for surgical image guidance, comprising:
receiving, by an image processing system, real-time image data of a patient from the first imaging device;
receiving, by the image processing system, secondary image data from a second imaging device;
producing, by the image processing system, composite image data based on the real-time image data and the secondary image data; and
producing, by the image processing system, enhanced composite image data by improving an alignment of physical structures in a real-time image based on said real-time image data with corresponding physical structures in a secondary image based on said secondary image data,
receiving, by the image processing system, a command to permit moving the real-time image relative to the secondary image when the image processing system is operating in an unlocked mode in which the real-time image is free to move relative to the secondary image, and
receiving, by the processing system, a command to put the processing system in a locked operating mode so as to prevent relative movement between the real-time image and the secondary image.

18. The method according to claim 17, further comprising allowing for correction, by the processing system, of a misalignment between the real-time image data relative to the secondary image data until an operator determines that the real-time image data coincides with the secondary image data.

19. The method according to claim 17, further comprising computing, by the processing system, an image registration quality (IRQ) metric continuously, the IRQ metric quantifying a degree of the alignment of the physical structures in the real-time image with the corresponding physical structures in the secondary image.

20. The method according to claim 19, wherein the IRQ metric is determined by performing a two-dimensional cross-correlation between the real-time image data and the corresponding secondary image data according to the following equation:
$${r}_{ij} = \frac{{\sum }_{m} {\sum }_{n} [ f ( m + i , n + j ) - \overline{f} ] [ g ( m , n ) - \overline{g} ]}{\sqrt{{\sum }_{m} {\sum }_{n} {[ f ( m , n ) - \overline{f} ]}^{2} {\sum }_{m} {\sum }_{n} {[ g ( m , n ) - \overline{g} ]}^{2}}}$$
where f(m,n) and g(m,n) are respective image pixel values in the real-time image data and corresponding secondary image data at locations m,n, with i=j=0 for determining said correlation at the current alignment pose.

21. The method according to claim 19, further comprising:
determining whether the IRQ metric meets a predetermined threshold value or a dynamically determined threshold value; and
providing, based on the determination, a feedback signal to an operator or to automatically put the image processing system in the locked mode to prevent relative movement between the real-time image and the secondary image.

22. The method according to claim 21, further comprising providing the IRQ metric as feedback to the operator.

23. The method according to claim 21, wherein the dynamically determined threshold value is a maximum or a minimum IRQ value.

24. The method according to claim 21, further comprising storing the IRQ metric and a corresponding alignment pose that achieved the IRQ metric.

25. The method according to claim 21, further comprising:
comparing a first IRQ metric obtained in a first attempted alignment pose with a second IRQ metric obtained in a second attempted alignment pose, and
storing as an alignment pose the first attempted alignment pose or the second attempted alignment pose that achieved a best IRQ value among the first IRQ metric and the second IRQ metric.

26. The method according to claim 25, wherein the best IRQ metric corresponds to a higher IRQ metric in the first and second IRQ metrics, a lower IRQ metric in the first and second IRQ metrics, or an IRQ metric among the first and second IRQ metrics that is closest to the predetermined threshold value.

27. The method according to claim 26, further comprising:
automatically locking an alignment between the physical structures in the real-time image with the corresponding physical structures in the secondary image, if, after a certain number of comparison iterations, no improvement in alignment is possible any more, or no improvement in alignment is expected any more based on a trend of the IRQ metric, or after the IRQ metric has reached the predetermined threshold value, and
selecting a previously stored alignment pose having a best achieved IRQ metric as an optimum alignment pose.

28. A non-transitory processor-readable medium having instructions stored thereon, which when executed by one or more processors, cause the one or more processors to implement a method comprising:
receiving, by an image processing system, real-time image data of a patient from the first imaging device;
receiving, by the image processing system, secondary image data from a second imaging device;
producing, by the image processing system, composite image data based on the real-time image data and the secondary image data; and
producing, by the image processing system, enhanced composite image data by improving an alignment of physical structures in a real-time image based on said real-time image data with corresponding physical structures in a secondary image based on said secondary image data,
receiving, by the image processing system, a command to enable moving the real-time image relative to the secondary image when the image processing system is operating in an unlocked mode in which the real-time image is free to move relative to the secondary image, and
receiving, by the processing system, a command to put the processing system in a locked operating mode so as to prevent relative movement between the real-time image and the secondary image.
